(19) United States
(12) Patent Application Publication
(10) Pub. No.: US 2003/0153011 A1
(43) Pub. Date: Aug. 14, 2003

(54) Methods and reagents for conducting multiplexed assays of multiple analytes

(76) Inventor: Michael L. Bell, Fullerton, CA (US)

Correspondence Address: PATENT LEGAL DEPARTMENT/A-42-C, BECKMAN COULTER, INC., 4300 N. HARBOR BOULEVARD, BOX 3100, FULLERTON, CA, 92834-3100, US

(21) Appl. No.: 10/071,395

(22) Filed: Feb. 8, 2002

(51) Int. Cl. G01N 33/53; G01N 33/542; G01N 33/552; C12Q 1/68

(52) U.S. Cl. 435/7.9; 435/6; 436/527

(57) ABSTRACT

The present invention relates to improved methods for conducting multiplexed assays of multiple target analytes in a manner that permits each target analyte to be assayed within a dynamic assay range. The invention further relates to reagents capable of implementing such methods.

### FIELD OF THE INVENTION

The present invention relates to improved methods for conducting multiplexed assays of multiple analytes in a manner that permits each target analyte to be assayed within a dynamic assay range for that analyte. The invention further relates to reagents capable of implementing such methods.

### BACKGROUND OF THE INVENTION

A broad range of ligand binding assay formats has been developed to permit protein-protein interactions, enzyme catalysis, small molecule-protein binding, and cellular functions to be efficiently assayed.

Such assays may be heterogeneous or homogeneous, and they may be sequential or simultaneous. Heterogeneous assays, which rely in part on the transfer of analyte from a liquid sample to a solid phase by the binding of the analyte during the assay to the surface of the solid phase are particularly employed. In heterogeneous assay techniques, the reaction product is separated from excess sample, assay reagents and other substances by removing the solid phase from the reaction mixture. At some stage of the assay, whose sequence varies depending on the assay protocol, the solid phase and the liquid phase are separated and the determination leading to detection and/or quantitation of the analyte is performed on one of the two separated phases. One type of solid phase that has been used are magnetic particles, which offer the combined advantages of a high surface area and the ability to be temporarily immobilized at the wall of the assay receptacle by imposition of a magnetic field while the liquid phase is aspirated, the solid phase is washed, or both. Descriptions of such particles and their use are found in Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992).

In order to eliminate the bound-free separation step and reduce the time and equipment needed for a chemical binding assay, homogeneous assay formats have been described. In such assays, one component of the binding pair may still be immobilized, however, the presence of the second component of the binding pair is detected without a bound-free separation (see, e.g., Bishop, J. E. et al., “A Flow Cytometric Immunoassay For Beta2-Microglobulin In Whole Blood,” J Immunol. Meth. 210:79-87 (1997)).

Assay formats may be designed to be either competitive or noncompetitive. U.S. Pat. Nos. 5,563,036; 5,627,080; 5,633,141; 5,679,525; 5,691,147; 5,698,411; 5,747,352; 5,811,526; 5,851,778 and 5,976,822 illustrate several different assay formats and applications.

In competitive binding assays, the assay is designed so that the amount of label present on the solid phase will vary inversely with the amount of analyte present in the test sample. International Patent publication WO9926067A1 (Watkins et al.) describes competitive assays that have been performed using particles to which are bound molecules of a binding protein (such as an antibody) specific for the analyte. During the assay, the sample and a quantity of labeled analyte, either simultaneously or sequentially, are mixed with the microparticles. By using a limited number of binding sites on the microparticles, the assay causes competition between the labeled analyte and the analyte in the sample for the available binding sites. Examples of competitive binding assays include: U.S. Pat. No. 4,401,764 (Smith); U.S. Pat. No. 4,746,631 (Clagett); U.S. Pat. No. 4,661,444 (Li); U.S. Pat. No. 4,185,084 (Mochida et al.); U.S. Pat. No. 4,243,749 (Sadeh et al.); European Patent Publication EP 177,191 (Allen); GB Patent No. 2,084,317 (Chieregatt et al.).

In general, binding assay formats comprise three distinguishable response ranges. Where the amount of analyte being assayed is within the dynamic range of the assay, the reported signal will be dependent upon the amount of analyte present. Where the amount of analyte exceeds the dynamic range of the assay, saturation will occur and the reported signal will not be indicative of the true analyte concentration. Likewise, where the amount of analyte present in the sample falls below the threshold of the assay's dynamic range, the assay may be insufficiently sensitive to the actual analyte concentration, and the reported signal will also not be indicative of the true analyte concentration.

Two approaches have conventionally been employed to address this problem. In the first, multiple dilutions or concentrations of a sample are made and then assayed for a defined time period and the results are evaluated against that of a “standard curve” of assay results obtained with analyte of varying but known concentration. In the second approach, an amount of sample is assayed for multiple times, and results falling within the dynamic range of the assay are used to calculate the analyte's concentration (see, for example, U.S. Pat. No. 5,306,468 (Anderson et al.), U.S. Pat. No. 6,212,291 (Wang et al.)). U.S. Pat. Nos. 6,270,695; 6,218,137; 6,139,782; 6,090,571 and 6,045,727 (Akhavan-Tafti, et al.) and U.S. Pat. Nos. 6,045,991; 5,965,736 and 5,772,926 (Akhavan-Tafti) indicate the possibility of making multiple exposures in chemiluminescent assays. The use of multiple exposures in photography is also known (see, for example, U.S. Pat. No. 6,177,958 (Anderson) and U.S. Pat. No. 5,754,229 (Elabd)).

Microtiter or multi-well plates are becoming increasingly popular in various chemical and biological assays. High-density format plates, such as 384, 864 and 1536 well plates, are beginning to displace 96-well plates as the plate of choice. Many of the assays conducted in multiwell plates employ some type of light detection from the plate as the reporter for positive or negative assays results. Such assays include fluorescence assays, chemiluminescence assays (e.g., luciferase-based assays), phosphorescence assays, scintillation assays, and the like. In particular, with the advent of solid phase scintillating materials, capsules and beads, homogeneous scintillation proximity assays (SPA) are now being performed with increasing frequency in multiwell plates. Detection of light signals from multiwell plates in the past has typically been done using plate readers, which generally employ a photodetector, an array of such photodetectors, photomultiplier tubes or a photodiode array to quantify the amount of light emitted from different wells (see, for example, U.S. Pat. No. 4,810,096 (Russell, et al.) and U.S. Pat. No. 5,198,670 (VanCauter, et al.)).

It is increasingly desirable to assay multiple different analytes simultaneously in the same sampling. Such “multiplexing” permits greater throughput, minimizes sample volume and handling, provides internal standardization control, decreases assay cost and increases the amount of information that is obtainable from each sample. A significant complexity arises, however, from the fact that the concentrations of the individual analytes being assayed may vary unpredictably. As a consequence, it is difficult to ensure that each analyte is being assayed within the dynamic range of the assay for that analyte. Thus, for some analytes being assayed, the assay conditions may fall outside of the dynamic range of the assay, thereby failing to produce reportable results.

Various approaches for conducting multiplexed assays have been proposed. U.S. Pat. No. 6,319,668 (Nova, et al.), for example, employs computer-facilitated microarrays of reagents to conduct multiplexed analysis of multiple analytes. International Patent publication WO9926067A1 (Watkins et al.) describes the use of magnetic particles that vary in size to assay multiple analytes; particles belonging to different distinct size ranges are used to assay for different analytes. The particles are designed to be distinguishable by flow cytometry. Vignali, D. A. A. has described an alternative multiplex binding assay in which 64 different bead sets of microparticles are employed, each having a uniform and distinct proportion of two dyes (Vignali, D. A. A., “Multiplexed Particle-Based Flow Cytometric Assays,” J. Immunol. Meth. 243:243-255 (2000)). A similar approach involving a set of 15 different beads of differing size and fluorescence has been disclosed as useful for simultaneous typing of multiple pneumococcal serotypes (Park, M. K. et al., “A Latex Bead-Based Flow Cytometric Immunoassay Capable Of Simultaneous Typing Of Multiple Pneumococcal Serotypes (Multibead Assay),” Clin Diagn Lab Immunol. 7:486-9 (2000)). Bishop, J. E. et al. have described a multiplex sandwich assay for simultaneous quantification of six human cytokines (Bishop, J. E. et al., “Simultaneous Quantification of Six Human Cytokines in a Single Sample Using Microparticle-based Flow Cytometric Technology,” Clin Chem. 45:1693-1694 (1999)).

Despite such methods for conducting the multiplexed analysis of multiple analytes (see U.S. Pat. No. 6,319,668 (Nova, et al)), a need remains for efficient methods capable of simultaneously assaying multiple different analytes. The present invention addresses this need, as well as other needs.

### SUMMARY OF THE INVENTION

The present invention relates to improved methods for conducting multiplexed assays of multiple analytes in a manner that permits each analyte to be assayed within a dynamic assay range for that analyte. The invention further relates to reagents capable of implementing such methods.

In its preferred embodiments, the invention concerns the use of porous or otherwise modified supports in order to alter the kinetic rate of binding between an analyte and a ligand capable of binding such analyte, and thus permits assays to be conducted within their dynamic range without a need to dilute the reactants. The invention thus achieves a “virtual” dilution, and can be readily employed in applications in which multiple target analytes are to be simultaneously assayed (e.g., multiplex applications).

In detail, the invention concerns a method for assaying one or more target analytes in a sample, wherein the method comprises:

(A) providing, for at least one target analyte to be assayed, a binding ligand of the target analyte, the binding ligand being bound to a solid support; wherein the ability of the binding ligand to bind to the target analyte is hindered by a steric interference that does not hinder the binding of all other target analyte(s) to all other binding ligand(s);

(B) determining, for such target analyte(s), the presence, absence, activity or concentration of the target analyte(s), by determining the extent of binding between the target analyte and the solid-support-bound binding ligand of the target analyte.

The invention particularly concerns the embodiment of such method, wherein the steric interference is provided by the solid support.

The invention also concerns a method for assaying one or more target analytes in a sample, wherein the method comprises:

(A) providing, for at least one target analyte to be assayed, a binding ligand of the target analyte, the binding ligand being bound to a solid support; wherein the support is porous and wherein binding ligand is bound to the support within the pores of the support and the pores sterically interfere with the ability of the binding ligand to bind to the target analyte and wherein the ability of the binding ligand to bind to the target analyte is hindered by a steric interference that does not hinder the binding of all other target analyte(s) to all other binding ligand(s);

(B) determining, for such target analyte(s), the presence, absence, activity or concentration of the target analyte(s), by determining the extent of binding between the target analyte and the solid-support-bound binding ligand of the target analyte.

The invention particularly concerns the embodiment of such methods, wherein the support is controlled pore glass or a porous polymeric material.

The invention also concerns a method for assaying one or more target analytes in a sample, wherein the method comprises:

(A) providing, for at least one target analyte to be assayed, a binding ligand of the target analyte, the binding ligand being bound to a solid support; wherein the support comprises bound interfering molecules that sterically interfere with the ability of the binding ligand to bind to the target analyte but does not hinder the binding of all other target analyte(s) to all other binding ligand(s);

(B) determining, for such target analyte(s), the presence, absence, activity or concentration of the target analyte(s), by determining the extent of binding between the target analyte and the solid-support-bound binding ligand of the target analyte.

The invention also concerns a method for assaying one or more target analytes in a sample, wherein the method comprises:

(A) providing, for at least one target analyte to be assayed, a binding ligand of the target analyte, the binding ligand being bound to a solid support; wherein the ability of the binding ligand to bind to the target analyte is hindered by a chemical interference that does not hinder the binding of all other target analyte(s) to all other binding ligand(s);

(B) determining, for such target analyte(s), the presence, absence, activity or concentration of the target analyte(s), by determining the extent of binding between the target analyte and the solid-support-bound binding ligand of the target analyte.

The invention particularly concerns the embodiment of such methods, wherein the chemical interference is provided by the solid support.

The invention further concerns the embodiment of such methods, wherein the support comprises a plasticized organic phase particle, and wherein the binding ligand is immobilized within the confines of such particle.

The invention additionally concerns a method for assaying one or more target analytes in a sample, wherein the method comprises:

(A) providing, for at least one target analyte to be assayed, a binding ligand of the target analyte, the binding ligand being bound to a solid support; wherein the support comprises bound interfering molecules that chemically interfere with the ability of the binding ligand to bind to the target analyte but which do not hinder the binding of all other target analyte(s) to all other binding ligand(s);

(B) determining, for such target analyte(s), the presence, absence, activity or concentration of the target analyte(s), by determining the extent of binding between the target analyte and the solid-support-bound binding ligand of the target analyte.

The invention further concerns the embodiment of such methods, wherein the interfering molecules hinder binding by presenting a partial barrier to binding by the target analyte, and/or wherein the interfering or competing molecules comprise a tethered chain of at least 5 carbon atoms.

The invention further concerns the embodiment of all such methods, wherein the determination of the extent of binding between a target analyte and a binding ligand of the solid support comprises incubating the solid support in the presence of a detectably labeled binding ligand-binding molecule (especially and determining the presence, absence, or concentration of detectably labeled binding ligand-binding bound to the solid-support-bound binding ligand of the target analyte.

The invention further concerns the embodiment of such methods wherein the detectable label of the detectably labeled binding ligand-binding molecule is a fluorescent label.

The invention further concerns the embodiment of such methods wherein the determination of the extent of binding between the target analyte and the binding ligand of the solid support the step (B) employs flow cytometry.

The invention additionally concerns a composition for assaying a target analyte, which comprises a binding ligand of the target analyte bound to a solid support, wherein the support provides a steric interference that hinders the ability of the target analyte to bind to the bound binding ligand.

The invention further concerns the embodiment of such composition wherein the support is porous and wherein binding ligand is bound to the support within the pores of the support and the pores sterically interfere with the ability of the binding ligand to bind to the target analyte.

The invention further concerns the embodiment of such composition wherein the support is controlled pore glass or a porous polymeric material.

The invention additionally concerns a composition for assaying a target analyte, which comprises a binding ligand of the target analyte bound to a solid support, wherein the support provides a chemical interference that hinders the ability of the target analyte to bind to the bound binding ligand.

The invention further concerns the embodiment of such compositions wherein the support comprises a plasticized organic phase particle, and wherein the binding ligand is immobilized within the confines of such particle.

The invention further concerns the embodiment of such compositions wherein the support comprises bound interfering molecules that interfere with the ability of the binding ligand to bind to the target analyte, and/or wherein the interfering molecules hinder binding by presenting a partial barrier to binding by the target analyte, and/or wherein the interfering molecules comprise a tethered chain of at least 5 carbon atoms.

The invention further concerns a kit for assaying a target analyte, which comprises:

(A) a first container containing a binding ligand of the target analyte bound to a solid support, wherein the support provides a steric or chemical interference that hinders the ability of the target analyte to bind to the bound binding ligand; and

(B) a second container containing a detectably labeled binding ligand-binding molecule.

The invention further concerns the embodiment of such kit wherein the detectable label is a fluorescent label.

### BRIEF DESCRIPTION OF THE FIGURES:

FIG. 1 illustrates the cross-section of a porous particle solid support of the present invention in which ligand molecules (shown as “*”) specific for a target analyte have been bound to sites in the pores of the particle.

FIG. 2 illustrates the cross-section of a particle solid support of the present invention in which ligand molecules (shown as “*”) specific for a target analyte have been bound to the surface of the particle, which has been treated with interfering molecules (shown as “T”) to hinder analyte-ligand interactions.

FIG. 3 illustrates the cross-section of a particle solid support of the present invention in which ligand molecules (shown as “*”) specific for a target analyte have been bound to the surface of the particle, which has then been treated by a coating to hinder analyte-ligand interactions.

FIG. 4 illustrates the cross-section of a particle solid support of the present invention in which ligand molecules (shown as “*”) specific for a target analyte have been immobilized within the confines of plasticized organic phase particles.

### DESCRIPTION OF THE PREFERRED EMBODIMENTS:

The present invention relates to improved methods and reagents for conducting multiplexed assays of multiple target analytes (i.e., assays of one or more, and more preferably, two or more target analytes) in a manner that permits each target analyte to be assayed within a dynamic assay range for that analyte. Most preferably, such assays involve causing the target analyte to become bound to a solid support via a binding interaction with a ligand of the target analyte under conditions in which such binding is hindered. As used herein, binding is said to be “hindered” if its rate or extent is decreased but not eliminated by such conditions.

I. Definitions

As used herein, the term “dynamic range” of an assay is intended to denote the concentration range of a target analyte in a sample in which the detected signal of the assay (or a change of such signal) is dependent upon the concentration of the target analyte. The dynamic ranges of different target analytes may thus be the same or different, and may be overlapping or distinct.

As used herein, the term “target analyte” is intended to denote a compound or compounds whose presence, absence or concentration are the object of the assay. The term “ligand” as used herein is intended to denote a compound or compounds that have the ability to bind to a particular target analyte without binding to other target analytes that may be present in the sample.

Virtually any compound can be employed as a target analyte or ligand in the present invention. Without limitation, such analytes or ligands may be enzymes, co-factors, receptors, receptor ligands, hormones, cytokines, blood factors, viruses, antigens, steroids, drugs, antibodies, etc. For example, where an analyte is an enzyme, the ligand can be a substrate, co-factor, etc. Likewise, where an analyte is an antigen, the ligand may be an antibody or other antigen-binding molecule. By way of illustration, the target analytes or ligands of the present invention may include:

enzymes or other proteins whose expression is characteristic of disease (e.g., bone specific alkaline phosphatase, aldose reductase, myoglobin, troponin I, etc.);

drugs or metabolites (e.g., anti-cancer drugs, chemotherapeutic drugs, anti-viral drugs, non-steroidal anti-inflammatory drugs (NSAID), steroidal anti-inflammatory drugs, anti-fungal drugs, detoxifying drugs, analgesics, bronchodilators, anti-bacterial drugs, antibiotic drugs, diuretics, digoxin, antimetabolites, calcium channel blockers, drugs for treatment of psoriasis, substances of abuse (e.g., cocaine, opiates, and other narcotics), pesticides, herbicides, etc.);

co-factors (including vitamins, such as vitamin B12, folate, T₃, T₄, TU, FT₃, FT₄, etc.);

cell-surface receptors (e.g., receptors for TNF and related factors (e.g., Trk, Met, Ron, Axl, Eph, Fas, TNFRI, TNFRII, CD40, CD30, CD27, 4-1BB, LNGFR, OX40), serine-threonine kinase receptors (e.g., TGFβR), transmembrane 7 or G protein-coupled receptor families (e.g., CCR1, CCR2α, β, CCR3, CCR4, CCR5, CXCR1, CXCR2, CXCR3, CXCR4, BLR1, BLR2, V28, and class I and class II cytokines), receptors such as CD4, class I (hematopoietic cytokine) receptors (e.g., IL-1β, IL-2R β and γ chains, IL-3Rα, IL-5Rα, GMCSFRα, the IL-3/IL-5/GM-CSF receptor common β-chain, IL-4Rα, IL-7Rα, IL-9Rα, IL-10R, IL-11Rα, IL-13Rα, LIFR β, TPOR, OBR, IL-6Rα, gp130, OSMRβ, GCSFR, IL-11Rα, IL-12Rb1 and IL-12Rb2, GHR, PRL, and EPO), EGFR, PDGFR, MCSFR, SCFR, insulin-R, VEGFR, and class II receptors (e.g., IFNgRα, IFNgRβ, IL-10R, tissue factor receptor (TFR), and IFNαR1), etc.);

hormones (such as adrenaline (epinephrine), adrenocorticotropic hormone (ACTH), androgens (e.g., testosterone), angiotensinogen, antidiuretic hormone (ADH) (vasopressin), atrial-natriuretic peptide (ANP), calciferol (vitamin D3), calcitonin, calcitriol, cholecystokinin, chorionic gonadotropin (CG), dopamine, erythropoietin, estrogens (e.g., estradiol), follicle-stimulating hormone (FSH), gastrin, glucagon, glucocorticoids (e.g., cortisol and urinary cortisol), gonadotropin-releasing hormone (GnRH), gorticotropin-releasing hormone (CRH), growth hormone (GH), growth hormone-releasing hormone (GHRH), insulin, insulin-like growth factor-1 (IGF-1), leptin, luteinizing hormone (LH), melatonin, mineralocorticoids (e.g., aldosterone), neuropeptide Y, noradrenaline (norepinephrine), oxytocin, parathyroid hormone (PTH), progesterone, prolactin (PRL), renin, secretin, somatostatin, theophylline, thiiodothyronine T3, thrombopoietin, thyroid-stimulating hormone (TSH), thyrotropin-releasing hormone (TRH), thyroxine (T4);

cytokines (such as the interleukins (e.g., IL-2, IL-3, IL-4, IL-5, IL-6, IL-7, IL-8, IL-10, IL-12, IL-13) or TNFα, VEGF, GMCSF, IL-1β, FGFβ, INFγ, EGF, PDGF, MCSF, SCF, insulin, VEGF, Trk, Met, Ron, Axl, Eph, Fas, CD40, CD30, CD27, 4-1BB, LNGFR, OX40, TGFβR, or a ligand of CCR1, CCR2β, β, CCR3, CCR4, CCR5, CXCR1, CXCR2, CXCR3, CXCR4, BLR1, BLR2, V28 receptor, or a receptor of IL-2, IL-3, IL-4, IL-5, IL-6, IL-7, IL-8, IL-10, IL-12, or IL-13;

antigens (such as those characteristic of Chlamydia, Streptococcus pyogenes Group A bacteria, H. pylori, or M. tuberculosi, hepatitis virus, rubella, CMV or immunodeficiency virus (HIV, FIV), prostate specific antigen, etc.); or

antibodies to such antigens, or autoimmune immunoglobulins, thyroglobulin, anti-thyroglobulin, IgE, IgG, or IgM immunoglobulins, tumor markers (e.g., prostate specific antigen, AFP CEA, etc.).

II. Embodiments of the Preferred Assays of the Invention

### A. Overview of the Principles of the Preferred Assays of the Invention

In multiplexed reaction systems, the concentration of sample to be employed is usually determined by the assay having the greatest sensitivity requirement. This is problematic for the measurement of analytes of high concentration in the same mixture.

The affinity with which a ligand binds to an analyte is related to the specificity of the interaction. Consequently, when solid phase-bound ligands are employed to assay a high concentration target analyte, a receptor with high affinity must be used in order to achieve appropriate specificity of binding. Since only a small amount of receptor will be present on the surface of such a support, the reaction equilibrium will be altered by the presence of a high concentration of analyte so that substantially all of the receptor is bound by analyte (see, Sato, H. et al., “Effect Of Pore Size Of Porous Bead Carriers Immobilizing Antibody On IgE Absorption,” J Biomed Mater Res. 20:853-8 (1986)). Accordingly, at equilibrium, the signal difference obtained from different analyte concentrations may be very small.

High concentrations of biological ligands in free solution rapidly approach equilibrium. For example, the Array immunonephelometric assay for IgG (Beckman Coulter, Inc.) is complete in less than one minute even though the sample forms less than one part in one thousand of the reaction mixture. In light of such kinetics, it is not practical to read multiplex binding results on a timescale short enough to avoid the range compression seen at equilibrium.

The invention addresses this problem by allowing measurement of high concentration target analytes in the same reaction mixture as low concentration target analytes. In preferred embodiments, the invention uses target analyte binding ligands bound to solid supports (especially beads) to capture the target analyte molecules. The quantity of captured target analyte is indicated by a second binding reaction that may occur in parallel with the capture reaction or in series with it. This second binding reaction preferably uses a delectably labeled “second” ligand-binding molecule that is able to bind to ligand molecules that have not become bound to target analyte molecules. Alternatively, the second binding reaction may employ a delectably labeled analyte-binding molecule that is able to bind to the bound target analyte molecules, so as to form a sandwich-like structure. The amount of label bound to the solid support is proportional to the concentration of target analyte in the sample.

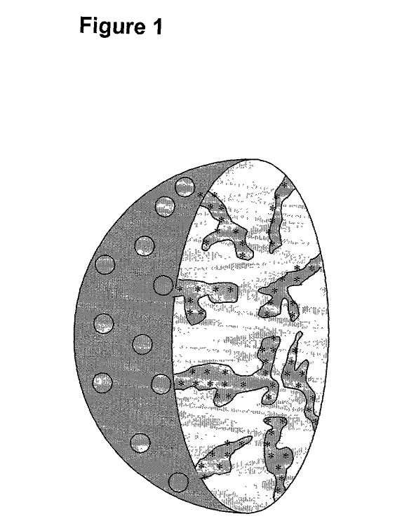

The present invention differs from prior binding assays in that it employs ligands that are sequestered to the solid support in such a way as to hinder the free diffusion of analyte among the ligands. In one embodiment, such sequestration is accomplished by immobilizing the ligand molecules within a sterically hindered environment, such as by employing a support comprising minute pores, and immobilizing the ligands within such pores (see FIG. 1).

The scale of the pores is preferably selected such that it is close to the size of large biological molecules such as IgG. As such, infiltrating molecules will frequently interact with the bead surface surrounding the porosities, thereby reducing the rate of diffusion of target analyte through the pores and hence the frequency of analyte-ligand collisions within the pores of the support. This characteristic reduces the rate of binding and extends the time needed to attain equilibrium (see, for example, Horstmann, B. J. et al., “Rate-Limiting Mass Transfer In Immunosorbents: Characterisation Of The Adsorption Of Paraquat-Protein Conjugates To Anti-Paraquat Sepharose 4B,” Bioseparation 7:145-57 (1998); Schmidt, D. E., Jr. et al., “An Advanced Solid Support For Immunoassays And Other Affinity Applications,” Biotechniques 14:1020-1025 (1993)). The extended time to equilibrium provided by the present invention permits the extent of the binding reaction to be determined within a practical time interval.

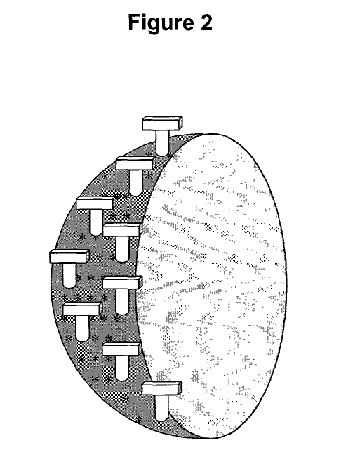

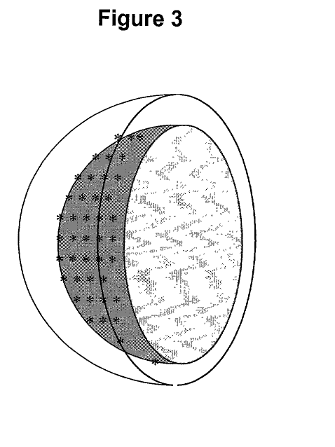

In an alternative embodiment, the ligand molecules may be bound to the surface of the support (as in conventional assays involving beads). In such embodiment, the support surface also has bound interfering or competing molecules that act to hinder binding by the target analyte and reduce the frequency of productive collisions between the ligand and target analyte (FIG. 2). Large interfering molecules may sterically hinder ligand—analyte binding. Other molecules may hinder binding by presenting a barrier to entry for the analyte. For example, a coating of tethered long-chain lipids may be used to create a local hydrophobic environment in which aqueous proteins would be only sparingly soluble (see FIG. 3). Long-chain lipids preferably have between 5 and 30 carbon atoms in the lipid chain, more preferably between 8 and 30 carbon atoms in the lipid chain.

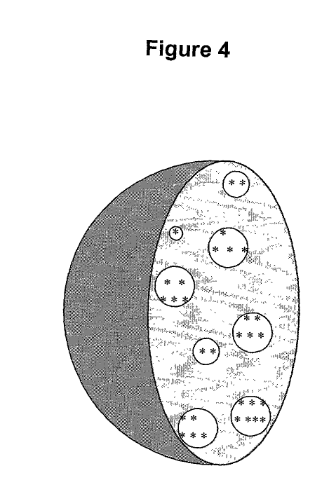

A further alternative involves immobilizing the ligand within the confines of plasticized organic phase particles such as produced by Beckman-Coulter, Inc. for electrolyte measurement (see U.S. Pat. No. 6,165,796). The target analyte must first enter the less energetically favorable semi-organic phase before it can bind to ligand (see FIG. 4).

Solid supports embodying combinations of any such embodiments can be employed (e.g., porous supports in which ligand is found at the surfaces as well as within the pores, or which have been treated to possess interfering molecules, etc.).

By hindering the diffusion of analyte to the ligand, the present invention extends the time needed to achieve equilibrium, and therefore expands the dynamic range of the assay via a virtual (rather than actual) dilution. Although such hindering may be accomplished in a variety of alternate ways (such as by increasing the viscosity of the medium with thickening agents or lowering the reaction temperature), such approaches act on the reaction as a whole and affect the signal from all analytes. Thus, in part, the present invention differs from conventional methods by hindering diffusion only in the vicinity of the support, by permitting different degrees of such interference with different target analytes, and by permitting some analytical reactions to proceed without interference.

The invention thus permits measurement of high concentration analytes in the same reaction mixture as low concentration analytes. This reduces the number of separate analyses necessary to complete a full clinical menu. Significantly, measurement does not require problematic low-affinity receptors and does not significantly affect other analyses in the reaction mixture. Significantly, the invention may be used to assay a single target analyte, or more than one target analyte (e.g., two or more target analytes) that may be present in a sample, in a manner that permits each target analyte to be assayed within a dynamic assay range for that analyte.

### B. Preferred Supports of the Invention

The supports of the present invention may comprise any of a variety of forms: beads, sheets, columns, etc. Most preferably, such supports will be bead-like spherical particles. In a preferred embodiment, such particles may be a controlled pore glass (CPG) bead (see, for example, Gormley, G. J. et al., “A Controlled Pore Glass Bead Assay For The Measurement Of Cytoplasmic And Nuclear Glucocorticoid Receptors,” J Steroid Biochem. 22:693-8 (1985)). CPG beads of 5 μm diameter with a nominal pore diameter of 50 nm have approximately 200 times more effective surface area than nonporous beads. Such increased surface area allows ligand attachment to the bead surface without any requirement for special precautions to prevent ligand molecule binding to the outer surface of the bead. The majority of ligand molecules bind to internal surfaces within the pores. Since ligand molecules bound to the outer surface represent only a small fraction of the total bound ligand, the rapidly saturated signal obtained from the binding of target analyte to surface-bound ligand molecules forms only a small proportion of the total signal generated. To reach the ligand molecules bound to the internal pores, the target analyte molecules must diffuse into the bead through its porous network.

CPG beads differ from polystyrene beads in that they do not have an interior readily accessible for coding dyes or other detectable labels. This problem may be dealt with by coupling the detectable labels to the particle surface inside the pores. If binding sites are limited, the detectable labels may be coupled to the capture receptors and that complex coupled to the pore surfaces. Alternatively, bifunctional detectable labels may be employed that possess two coupling sites. The first of such sites would permit attachment of the label to the particle; the second of such sites would be used to couple a ligand molecule to the bound label.

Alternatively, such particles may comprise a polymeric material. Such polymeric material can be any material that can be formed into a microparticle that does not adversely interfere with the assay. Examples of suitable polymers are agaroses, polyesters, polyethers, polyolefins, polyalkylene oxides, polyamides, polyurethanes, polysaccharides, celluloses, polyisoprenes and acrylamides. Crosslinking is useful in many polymers for imparting structural integrity and rigidity to the microparticle, or controlling pore size. Hydrophilic acrylamide is a preferred material.

Functional groups suitable for facilitating the attachment of the ligand can be incorporated into the polymer structure by conventional means, including the use of monomers that contain the desired functional group(s), either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), isocyanate groups (—NCO), etc. A useful monomer for introducing carboxylic acid groups into polyolefins, for example, is acrylic acid or methacrylic acid.

Attachment of the ligand to the microparticle can be achieved by electrostatic attraction, specific affinity interaction, hydrophobic interaction, or covalent bonding. Covalent bonding is preferred. Linking groups can be used as a means of increasing the density of reactive groups on the microparticle and of modulating steric hindrance to increase the range and sensitivity of the assay, or as a means of adding specific types of reactive groups to the microparticle to broaden the number of types of ligands that can be affixed to the microparticle. Examples of suitable useful linking groups are polylysine, polyaspartic acid, polyglutamic acid, polyarginine, etc.

### C. Preferred Assay Formats

Any of a wide variety of assay formats may be used in accordance with the methods of the present invention. They may be heterogeneous or homogeneous, and they may be sequential or simultaneous. They may be competitive or noncompetitive. U.S. Pat. Nos. 5,563,036; 5,627,080; 5,633,141; 5,679,525; 5,691,147; 5,698,411; 5,747,352; 5,811,526; 5,851,778 and 5,976,822 illustrate several different assay formats and applications.

Most preferably, however, the assay will involve a heterogeneous format involving the use of a solid phase material to which the target analyte becomes bound. The reaction product is separated from excess sample, assay reagents and other substances by removing the solid phase from the reaction mixture.

In order to eliminate the bound-free separation step and reduce the time and equipment needed for a chemical binding assay, a homogeneous assay format may be used. In such assays, one component of the binding pair may still be immobilized, however, the presence of the second component of the binding pair is detected without a bound-free separation. Examples of homogeneous, optical methods are the EMIT method of Dade Behring, Inc. (Deerfield, Ill.), which operates through detection of fluorescence quenching, the laser nephelometry latex particle agglutination method of Dade Behring, Inc., which operates by detecting changes in light scatter, the LPIA latex particle agglutination method of Mitsubishi Chemical Industries, the TDX fluorescence depolarization method of Abbott Laboratories (Abbott Park, Ill.), and the fluorescence energy transfer method of Cis Bio International (Paris, France). Any of such assays may be employed in accordance with the present invention.

The binding assay of the present invention may be configured as a competitive assay. In a competitive assay, the more analyte present in the test sample the lower the amount of label present on the solid phase. In a manner similar to the sandwich assay, the competitive assay can involve an anti-analyte binding agent bound to the insoluble solid phase, however, a labeled analyte, instead of a labeled second antibody of the sandwich assay, is used as the indicator reagent. In the competitive assay, the indicator reagent competes with the test sample analyte to bind the capture reagent on the solid phase. The amount of captured indicator reagent is inversely related to the amount of analyte present in the test sample. Smith (U.S. Pat. No. 4,401,764) describes an alternative competitive assay format using a mixed binding complex which can bind analyte or labeled analyte but wherein the analyte and labeled analyte cannot simultaneously bind the complex. Clagett (U.S. Pat. No. 4,746,631) describes an immunoassay method using a reaction chamber in which an analyte/ligand/marker conjugate is displaced from the reaction surface in the presence of test sample analyte and in which the displaced analyte/ligand/marker conjugate is immobilized at a second reaction site. The conjugate includes biotin, bovine serum albumin and synthetic peptides as the ligand component of the conjugate, and enzymes, chemiluminescent materials, enzyme inhibitors and radionucleotides as the marker component of the conjugate. Li (U.S. Pat. No. 4,661,444) describes a competitive immunoassay using a conjugate of an anti-idiotype antibody and a second antibody, specific for a detectable label, wherein the detectable response is inversely related to the presence of analyte in the sample. Allen (EP 177,191) describes a binding assay involving a conjugate of a ligand analog and a second reagent, such as fluorescein, wherein the conjugate competes with the analyte (ligand) in binding to a labeled binding partner specific for the ligand, and wherein the resultant labeled conjugate is then separated from the reaction mixture by means of solid phase carrying a binding partner for the second reagent. This binding assay format combines the use of a competitive binding technique and a reverse sandwich assay configuration, i.e., the binding of conjugate to the labeled binding member prior to separating conjugate from the mixture by the binding of the conjugate to the solid phase. The assay result, however, is determined as in a conventional competitive assay wherein the amount of label bound to the solid phase is inversely proportional to the amount of analyte in the test sample. Chieregatt et al. (GB Patent No. 2,084,317) describe a similar assay format using an indirectly labeled binding partner specific for the analyte. Mochida et al. (U.S. Pat. No. 4,185,084) also describe the use of a double-antigen conjugate which competes with an antigen analyte for binding to an immobilized antibody and which is then labeled; this method also results in the detection of label on a solid phase wherein the amount of label is inversely proportional to the amount of analyte in the test sample. Sadeh et al. U.S. Pat. No. 4,243,749) describe a similar enzyme immunoassay wherein a hapten conjugate competes with analyte for binding to an antibody immobilized upon a solid phase. Any of such variant assays may be used in accordance with the present invention.

In all such assay formats, at least one of the components of the assay reagents will be labeled or otherwise detectable by the evolution or quenching of light. Such component may be the analyte being assayed, or a substrate, co-factor, binding partner, or product of a reaction or activity of such analyte, etc. Radioisotopic-binding assay formats (e.g., a radioimmunoassay, etc.) employ a radioisotope as such label; the signal being detectable by the evolution of light in the presence of a fluorescent or fluorogenic moiety (see, U.S. Pat. No. 5,698,411 (Lucas, et al.) and U.S. Pat. No. 5,976,822 (Landrum et al.). Enzymatic-binding assay formats (e.g., an ELISA, etc.) employ an enzyme as a label; the signal being detectable by the evolution of color or light in the presence of a chromogenic or fluorogenic moiety. Other labels, such as paramagnetic labels, materials used as colored particles, latex particles, colloidal metals, such as selenium and gold, and dye particles may also be employed (see U.S. Pat. Nos. 4,313,734; 4,373,932, and 5,501,985).

### D. Preferred Methods for Assay Signal Evolution

The present invention comprises a method to assay multiple target analytes simultaneously within the dynamic ranges of their respective binding assays. In a preferred embodiment, such binding assays will involve the evolution of a detectable fluorescent, chemiluminescent, calorimetric, radiological, nephelometric, turbidometric, ultraviolet, or other signal in response to the presence or absence of the target analyte. In a further embodiment, the presence, absence, or concentration of a target analyte will be assayed by a change (i.e., by the evolution or loss) of a light signal in two or more time intervals.

As used herein, the term “change” of a detectable signal is intended to include both processes resulting in an increase in signal (for example, as when a fluorescent product is produced over time as a consequence of the action of a target enzyme) as well as processes resulting in a decrease in signal (for example, as when a fluorescent substrate is consumed over time as a consequence of the action of a target enzyme). In accordance with the methods of the present invention, the detected light signal may involve light of the visible, near-UV, or UV wavelengths, and may be generated by chemiluminescent, fluorescent (including laser induced fluorescent), calorimetric, radiological, nephelometric, turbidometric or other mechanism (for example through the use of “second” ligand-binding molecules (or analyte-binding molecules) that emit or quench such light signal in response to the presence, absence or concentration of the target analyte).

Any of a wide variety of labels may be used in accordance with the principles of the present invention in order to generate such light signal. In one embodiment, such labels will possess a chemiluminescent moiety. Suitable chemiluminescent moieties include acridinium esters, ruthenium complexes, metal complexes (e.g., U.S. Pat. Nos. 6,281,021; 5,238,108 and 5,310,687), oxalate ester-peroxide combination, etc.)

Alternatively, such labels may possess a calorimetric moiety. Suitable calorimetric moieties include thiopeptolides, anthroquinone dyes, 2 methoxy 4(2 nitrovinyl) phenyl β-2 acetamido 2 deoxy βD glucopyranoside; ammonium 5[4(2 acetamido 2 deoxy βD glucopyranosyloxy) 3 methoxy phenylmethylene] 2 thioxothiazolin 4 one 3 ethanoate hydrate; 4{2[4(βD glucosyl pyranosyloxy) 3 methoxy phenyl]vinyl} 1 methylquinolinium iodide, 2 methoxy 4(2 nitrovinyl) phenyl βD galactopyranoside, 2{2[4(βD galactopyranosyloxy)3 methoxyphenyl]vinyl} 1 methyl quinolinium iodide, 2{2[4(βD galactopyranosyloxy)3 methoxyphenyl]vinyl} 3 methyl benzothiazolium iodide, 2{2[4(βD glucopyranosyloxy) 3 methoxyphenyl]vinyl} 1 methyl quinolinium iodide, 2{2[4(βD glucopyranosyloxy) 3 methoxyphenyl]vinyl} 1 propyl quinolinium iodide, 2{2[4(βD glucopyranosyloxy) 3 methoxyphenyl]vinyl} 3 methyl benzothiazolium iodide, ammonium 5[4βD glucopyranosyloxy) 3 methoxy phenylmethylene] 2 thioxothiazolin 4 one 3 ethanoate hydrate, 2 methoxy 4(2 nitrovinyl) phenyl acetate, 2 methoxy 4(2 nitrovinyl) phenyl propionate, 5[4 propanoyloxy) 3,5 dimethoxy phenylmethylene] 2 thioxothiazolin 4 one 3 ethanoate, 5[4 butanoyloxy) 3,5 dimethoxy phenylmethylene] 2 thioxothiazolin 4 one 3 ethanoate, 5[4 decanoyloxy) 3,5 dimethoxy phenylmethylene] 2 thioxothiazolin 4 one 3 ethanoate, 5[4 dodecanoyloxy) 3,5 dimethoxy phenylmethylene] 2 thioxothiazolin 4 one 3 ethanoate, 5[4 tetradecanoyloxy) 3,5 dimethoxy phenylmethylene] 2 thioxothiazolin 4 one 3 ethanoate, Pyridinium 4{2[4(phosphoroyloxy) 3,5 dimethoxyphenyl]vinyl} 1 propyl quinolinium iodide, Pyridinium 5(4 phosphoryloxy 3,5 dimethoxy phenylmethylene) 3 methyl 2 thioxothiazolin 4 one, etc.

Preferably, however, the detected light will be fluorescent, and the label will possess a fluorescence-generating moiety whose fluorescence is dependent upon the presence, absence or concentration of the target analyte. Examples of suitable fluorescence-generating moieties include rhodamine 110; rhodol; coumarin or a fluorescein compound. Derivatives of rhodamine 110, rhodol, or fluorescein compounds that have a 4′ or 5′ protected carbon may likewise be employed. Preferred examples of such compounds include 4′(5′)thiofluorescein, 4′(5′)-aminofluorescein, 4′(5′)-carboxyfluorescein, 4′(5′)-chlorofluorescein, 4′(5′)methylfluorescein, 4′(5′)-sulfofluorescein, 4′(5′)-aminorhodol, 4′(5′)carboxyrhodol, 4(5′)-chlororhodol, 4′(5′)-methylrhodol, 4′(5′)-sulforhodol; 4(5′)-aminorhodamine 110, 4′(5′)-carboxyrhodamine 110, 4′(5′)-chlororhodamine 110, 4′(5′)-methylrhodamine 110, 4′(5′)-sulforhodamine 110 and 4′(5′)thiorhodamine 110. “4′(5′)” means that at the 4 or 5′ position the hydrogen atom on the carbon atom is substituted with a specific organic group or groups as previously listed. A 7-Amino, or sulfonated coumarin derivative may likewise be employed. Any of a variety of cyanine dyes, such as those disclosed in U.S. Pat. Nos. 2,734,900, 6,002,003, or 6,110,630 may likewise be employed.

In a further embodiment, cellprobe reagents may be employed as the label. In general such cellprobe reagents contain an “indicator group” and one, two, three, four or even more “leaving groups.” The “indicator group” of the compound is a chemical moiety selected for its ability to have a first state when joined to the leaving group, and a second state when the leaving group is cleaved from the indicator group by the enzyme. The indicator group is preferably excitable (caused to fluoresce) at a wavelength about the visible range, for example, at wavelength between about 450 to 500 nanometers (nm). The indicator group will usually emit in the range of about 480 to 620 nm, preferably 500 to 600 nm and more preferably 500 to 550 nm. Auto-fluorescence of the cell is most prevalent below about 500 nm. The indicator group is preferably derived from fluorescent, calorimetric, bioluminescent or chemiluminescent compounds. The indicator group is preferably quenched when joined to the leaving group. The term quenched means that the indicator group has substantially less fluorescence or chemiluminescence when joined to the leaving group compared to its fluorescence or chemiluminescence after the leaving group has been cleaved. For example, the enzyme glutamyltranspeptidase reacts with gammaglutamyl amino acid peptide giving gamma glutamic acid; trypsin cleaves the peptide at the arginine residue; aminopeptidase-M hydrolyzes the peptide at the aliphatic amino acid residue; and chymotrypsin cleaves the peptide at the phenylalanine residue. Suitable fluorogenic indicator compounds include xanthine compounds. Preferably, the indicator compounds are rhodamine 110; rhodol; fluorescein; and coumarin, and their derivatives. While, for convenience, the invention is described below with respect to fluorescent leaving groups, it will be appreciated that the leaving groups may alternatively be enzymatic, colorimetric, bioluminescent, chemiluminescent, paramagnetic, luminescent, radioactive, etc.

Each “leaving group” of the compound is a chemical moiety selected so that it will be cleaved by the enzyme to be analyzed. For such embodiment, compounds having a molecular weight of less than about 5,000 are preferred. The leaving group is selected according to the enzyme that is to be assayed. The leaving group will preferably have utility for assaying any of a variety of cellular enzymes, including proteases, caspases, glycosidases, glucosidases, carbohydrases, phosphodiesterases, phosphatases, sulfatases, thioesterases, pyrophosphatases, lipases, esterases, nucleotidases and nucleosidases, as listed above.

The leaving group is preferably selected from amino acids, peptides, saccharides, sulfates, phosphates, esters, phosphate esters, nucleotides, polynucleotides, nucleic acids, pyrimidines, purines, nucleosides, lipids and mixtures thereof. For example, a peptide and a lipid leaving group can be separately attached to a single assay compound such as rhodamine 110. Other leaving groups suitable for the enzyme to be assayed can be determined empirically or obtained from the literature. See, for example, Mentlein, R. et al., H. R., “Influence of Pregnancy on Dipeptidyl Peptidase IV Activity (CD26 Leukocyte Differentiation Antigen) of Circulating Lymphocytes”, Eur. J. Clin. Chem. Clin. Biochem., 29, 477-480 (1991); Schon, E. et al., Eur. J. Immunol., 17, 1821-1826 (1987); Ferrer-Lopez, P. et al., “Heparin Inhibits Neutrophil-Induced Platelet Activation Via Cathepsin”, J. Lab Clin. Med. 119(3), 231-239 (1992); and Royer, G. et al., “Immobilized Derivatives of Leucine Aminopeptidase and Aminopeptidase M.”, J. Biol. Chem. 248(5), 1807-1812 (1973). These references are hereby incorporated by reference in their entirety.

Examples of such regents include (Cbz-Phe-Arg-NH)₂-rhodamine and (Cbz-Pro-Arg-NH)₂-rhodamine, which have particularly use in assays for human plasmin and human thrombin, respectively (Leytus, S. P. et al., “New class of sensitive and selective fluorogenic substrates for serine proteases,” Biochem. J. 215:253-260 (1983)).

Derivatives of the tetrapeptides ala-ala-pro-leu and ala-ala-pro-val (Beckman Coulter, Inc.) are preferred assay compounds for assaying the activity of the closely related enzymes leukocyte elastase and pancreatic elastase (leukocyte elastase is also known as neutrophil elastase, EC 3.4.21.37; pancreatic elastase is also known as EC 3.4.21.36) (Stein, R. L. et al. 1987, “Catalysis by human leukocyte elastase: Mechanistic insights into specificity requirements,” Biochem. 26:1301-1305; Stein, R. L. et al. 1987, “Catalysis by human leukocyte elastase: Proton inventory as a mechanistic probe,” Biochem. 26:1305-1314). Elastases are defined by their ability to cleave elastin, the matrix protein that gives tissues the property of elasticity. Human leukocyte elastase is a serine protease that is a major component of neutrophil granules and is essential for defense against infection by invading microorganisms (Bode, W. et al. 1989, “Human leukocyte and porcine pancreatic elastase: X-ray crystal structures, mechanism, substrate specificity and mechanism-based inhibitors,” Biochem. 28:1951-1963)

Aspartic acid-Rho110 (Beckman Coulter, Inc.) is a preferred assay compound for assaying the activity of the Ca-dependent enzyme aminopeptidase A (aspartate aminopeptidase, angiotensinase A, EC 3.4.11.7). Aminopeptidase A is found in both soluble and membrane-bound forms. Aminopeptidase A is known to cleave the N-terminal aspartic acid amino acid of angiotensin I or II (Jackson, E. K. et al., 1995, “Renin and Angiotensin” in Goodman and Gilman's The Pharmacological Basis of Therapeutics, Ninth Edition McGraw-Hill, N.Y.). Aminopeptidase A is also identical to BP-1/6C3 (Wu, Q. et al., 1991. “Aminopeptidase A activity of the murine B-lymphocyte differentiation antigen BP-1/6C3,” Proc. Natl. Acad. Sci, USA. 88: 676-680), a molecule found on early lineage B cells but not on mature lymphocytes. BP-1/6C3 may have a role in the ability to support long-term growth of B cells (Whitlock, C. A., et al., 1987. “Bone marrow stromal cell lines with lymphopoietic activity express high levels of a pre-B neoplasia-associated molecule,” Cell 48: 1009-1021.

The conversion of non-fluorescent dichlorofluorescein diacetate (DCFH-DA) (Beckman Coulter, Inc.) to the highly fluorescent compound 2′,7′-dichlorofluorescein (DCF) is a preferred assay compound for monitoring the oxidative burst in polymorphonuclear leukocytes and for determining the presence of peroxides formed through such oxidative bursts (Bass, D. A. et al. “Flow cytometric studies of oxidative product formation by neutrophils: a graded response to membrane stimulation.” J. Immunol. 130: 1910-1917). The enzymes responsible for the oxidative burst are rapidly activated in stimulated neutrophils (Weiss, S. J. 1989, “Tissue destruction by neutrophils,” N. Eng. J. Med. 320: 365-376). DCFH,PMA Oxidative Burst contains the compound phorbol myristate acetate (PMA), an analogue of the cellular signaling molecule diacylglycerol (DAG) (Alberts, B. et al., Molecular Biology of the Cell, 2nd Edition. Garland Publishing, Inc. N.Y., pg 704). Therefore, the presence of PMA stimulates processes mediated by DAG, including the oxidative burst. Additionally, resting cells do not have free peroxides and the production of peroxides is rapidly activated by many cell stimuli including the presence of the bacteria or other foreign organisms (Weiss. S. J. 1989, “Tissue destruction by neutrophils,” N. Eng. J. Med. 320: 365-376). The production of peroxides due to the oxidative burst can by artificially stimulated by the addition of the compound phorbol myristate acetate (PMA) to the neutrophils (CellProbe substrate DCFH, PMA Oxidative Burst). DCFH.Peroxides can be used to investigate the effect of other compounds on the oxidative burst including the chemotactic peptide f-met-leu-phe and the yeast product zymosan.

Fluorescein diacetate (FDA) (Beckman Coulter, Inc.) is a preferred assay compound for assaying the activity of many different non-specific esterases in human tissues (Coates, P. M. et al., 1975, “A preliminary genetic interpretation of the esterase isozymes of human tissues,” Ann. Hum. Genet. Lond. 39: 1-20). Acetate esterase activity measured with—Napthyl acetate has been used together with other esterase activities to identify leukocyte cell types and is generally high in normal monocytes and megakaryocytes and in blast cells of acute myelomonocytic leukemia, acute monocytic leukemia and acute erythroleukemia. Nelson, D. A. et al., 1990, “Leukocyte esterases in Hematology,” 4th Edition, Williams, Beutler, Erslev and Lichtman, Eds. McGraw-Hill.

Fluorescein di-galactopyranoside (Beckman Coulter, Inc.) is a preferred assay compound for assaying the activity of the galactosidase enzymes (β-galactosidase is also known as lactase, β-D-galactoside galactohydrolase, EC 3.2.1.23; α-galactosidase is also known as melibiase, α-D-galactoside galactohydrolase, EC 3.2.1.22) (Jongkind, J. F. et al., 1986, “Detection of acid-b-galactosidase activity in viable human fibroblasts by flow cytometry,” Cytometry 7:463-466). Galactosidase enzymes are lysosomal enzymes that cleave terminal sugar residues from several physiological substrates, including glycoproteins. Gal. galactosidase contains forms of the substrate that are hydrolyzed by both b-galactosidase and a-galactosidase. Impaired galactosidase activity leads to accumulation of partially digested glycoproteins in the lysosomes (Cotran, R. S. et al., 1994, Robbins Pathologic Basis of Disease, 5th Edition. W. B. Saunders Co. pages 138-140). The lysosomes become enlarged, and disrupt normal cell function. The impaired galactosidase activity may be due to mutations in the galactosidase genes or in the processing and transport mechanisms of galactosidase to the lysosomes.

Glycine-phenylalanine-glycine-alanine-Rho110 (Beckman Coulter, Inc.) is a preferred assay compound for assaying the activity of the collagenase group of proteolytic enzymes in a screen of several tetrapeptide derivatives. Collagenases are enzymes that digest the collagens: macromolecules that form highly organized structures in connective tissue and extracellular matrix. Collagenases and other members of the matrix metalloproteinase family contribute to physiological processes such as postpartum involution of the uterus, wound healing, joint destruction in arthritis, tumor invasion and periodontitis. The collagenases are Zn+2 dependent metallo-enzymes that are synthesized in a pro-enzyme inactive form (Woessner, J F Jr. 1991. Matrix metalloproteinases and their inhibitors in connective tissue remodeling. FASEB J. 5: 2145-2154). The production of HOCl during the neutrophil oxidative burst has been postulated as one mechanism for collagenase activation in vivo.

The assay compound, fluorescein di-glucuronide (Beckman Coulter, Inc.) is hydrolyzed by the lysosomal enzyme b-glucuronidase (β-glucuronidase is also known as β-D-glucuroniside glucuronosohydrolase, EC 3.2.1.31). A derivative of β-glucuronide has been used to measure degranulation in polymorphonuclear lymphocytes (PMNs) in a test of the ability of different non-steroidal anti-inflammatory drugs (NSAIDS) to inhibit PMN functions (Kankaanranta, H. et al., 1994, “Effects of non-steroidal anti-inflammatory drugs on polymorphonuclear leukocyte functions in vitro: focus on fenamates,” Naunyn-Schmiedeberg's Arch Pharmacol. 350:685-691). Peripheral blood T-lymphocytes display higher β-glucuronidase activity that peripheral blood B-lymphocytes (Crockard, A. et al., 1982, “Cytochemistry of acid hydrolases in chronic B- and T-cell leukemias,” Am. J. Clin. Pathol. 78:437-444). Fluorescein di-glucuronide is a negatively charged compound. To help other derivatives of sugars pass through cell membranes in assays of β-glucosidase, a lysomotropic detergent (N-dodecylimidazole) was used (Kohen, E. et al., 1993, “An in situ study of beta-glucosidase activity in normal and gaucher fibroblasts with fluorogenic probes,” Cell Biochem. and Function. 11:167-177).

Glycine-proline-Rho110 (Beckman Coulter, Inc.) is a preferred assay compound for assaying the activity of the serine protease dipeptidyl peptidase IV (DPP IV; Xaa-Pro-dipeptidyl-aminopeptidase, Gly-pro naphthylamidase, EC 3.4.14.5). The membrane bound form of DPP IV is also known as the T-cell activation cell surface marker CD26 (Fleischer, B., 1994, “CD26: a surface protease involved in T-cell activation,” Immunol. Today. 15: 180-184). The proteolytic activity of DPP IV may play an essential role in the signaling function of CD26 (Hegen, M. et al., 1993, “Enzymatic activity of CD26 (dipeptidylpeptidase IV) is not required for its signalling function in T cells,” Immunobiology 189: 483-493; Tanaka, T. et al., 1993, “The costimulatory activity of the CD26 antigen requires dipeptidyl peptidase IV enzymatic activity,” Proc. Natl. Acad. Sci. USA. 90: 4586-4590). DPP IV cleaves the N-terminal dipeptide from oligopeptides with sequences analogous to the N-terminal sequence of signaling molecules IL-1b, IL-2 and TNF-b, but does not have activity against intact recombinant molecules (Hoffmann, T. et al. 1993, “Dipeptidyl peptidase IV (CD 26) and aminopeptidase N (CD 13) catalyzed hydrolysis of cytokines and peptides with N-terminal cytokine sequences,” FEBS Letters. 336: 61-64). Studies of dipeptidyl peptidase IV activity with GP.DPP IV suggest that DPP IV is upregulated in mature thymocytes and among thymocytes which are undergoing programmed cell death (apoptosis) (Ruiz, P. et al., 1996, “Cytofluorographic evidence thatthymocyte dipeptidyl peptidase IV (CD26) activity is altered with stage of ontogeny and apoptotic status,” Cytometry. 23: 322-329.

Glycine-proline-leucine-glycine-proline-Rho110 (Beckman Coulter, Inc.) is a preferred assay compound for assaying the activity of the collagenase group of proteolytic enzymes. Collagenases are Zn+2 dependent metallo-enzymes that are synthesized in a pro-enzyme inactive form 1. (Collagenases digest the collagens: macromolecules that form highly organized structures in connective tissue and extracellular matrix. Collagenases and other members of the matrix metalloproteinase family contribute to physiological processes such as postpartum involution of the uterus, wound healing, joint destruction in arthritis, tumor invasion and periodontitis (Woessner, J. F. Jr., 1991, “Matrix metalloproteinases and their inhibitors in connective tissue remodeling,” FASEB J. 5: 2145-2154). In a detailed study of the mechanism of hydrolysis of fluorescent derivatives of GPLGP, Kojima et al. found that a collagenase-like peptidase cleaved the substrate at the peptide bond between leu and gly (Kojima, K. et al., 1979, “A new and highly sensitive fluorescence assay for collagenase-like peptidase activity,” Anal. Biochem. 100: 43-50).

Lys-Rho110 (Beckman Coulter, Inc.) is a preferred assay compound for assaying the activity of aminopeptidase B (EC 3.4.11.6). The aminopeptidases are a group of enzymes which hydrolyze peptide bonds near the N-terminus of polypeptides (International Union of Biochemistry and Molecular Biology. Enzyme Nomenclature. 1992. Academic Press, San Diego). Aminopeptidase B has been purified from the cytosolic fraction of human liver and skeletal muscle and shown to act on synthetic lysyl- or arginyl-substrates. Aminopeptidase B is activated by Cl-1 or Br-1 ions and inhibited by chelating agents and bestatin (Sanderink, G. J. et al., 1988, “Human Aminopeptidases: A Review of the Literature. J. Clin. Chem. Clin. Biochem. 26: 795-807).

Fluorescein di-phosphate (Beckman Coulter, Inc.) is a preferred assay compound for assaying the activity of the enzyme acid phosphatase (Acid phosphatase is also known as EC 3.1.3.2) (Rotman, B. et al., 1963, “Fluorogenic substrates for b-D-galactosidases and phosphatases derived from fluorescein (3,6- dihydroxyfluoran) and its monomethyl ether,”. Proc. Nat. Acad. Sci. USA 50:1-6). Assays of acid phosphatase activity have been used together with assays of esterase activity to identify many different cell types. Monocytes, neutrophils and T-lymphocytes have relatively high acid phosphatase activity while B-lymphocytes have relatively low acid phosphatase activity. (Crockard, A. et al., 1982, “Cytochemistry of acid hydrolases in chronic B- and T-cell leukemias,” Am. J. Clin. Pathol. 78:437-444; Li, C. Y. et al., 1970, “Acid phosphatase isoenzyme in human leukocytes in normal and pathologic conditions,” J. Histochem. Cytochem. 18:473-481). In addition, blast cells of acute promyelocytic leukemia and acute myelomonocytic leukemia have been shown to have relatively high acid phosphatase activity (Nelson, D. A. et al. 1990, “Leukocyte esterases in Hematology Fourth Edition,” Williams W J, Beutler E, Erslev A J and Lichtman MA eds. McGraw Hill, N.Y.

Arginine-Rho110 (Beckman Coulter, Inc.) is a preferred assay compound for assaying the activity of aminopeptidase B (arginyl aminopeptidase, EC 3.4.11.6). The aminopeptidases are a group of enzymes which hydrolyze peptide bonds near the N-terminus of polypeptides (International Union of Biochemistry and Molecular Biology. Enzyme Nomenclature. 1992. Academic Press, San Diego). Aminopeptidase B has been purified from the cytosolic fraction of human liver and skeletal muscle and shown to act on synthetic lysyl- or arginyl-substrates. Aminopeptidase B is activated by Cl-1 or Br-1 ions and inhibited by chelating agents and bestatin (Sanderink, G. J. et al., 1988, “Human Aminopeptidases: A Review of the Literature,” J. Clin. Chem. Clin. Biochem. 26: 795-807.

Arg-Gly-Glu-S er-Rho110 (Beckman Coulter, Inc.) is a preferred assay compound for assaying the activity of the closely related enzymes leukocyte elastase and pancreatic elastase (leukocyte elastase: neutrophil elastase, EC 3.4.21.37 pancreatic elastase: EC 3.4.21.36). Leukocyte elastase is a serine protease that is a major component of neutrophil granules and is essential for phagocytosis and defense against infection by invading microorganisms (Bode, W. et al., 1989, “Human leukocyte and porcine pancreatic elastase: X-ray crystal structures, mechanism, substrate specificity and mechanism-based inhibitors,” Biochem. 28: 1951-1963). The tetrapeptide RGES is part of the sequence of fibronectin (Gartner, T. K. et al., 1985, “The tetrapeptide analogue of the alpha chain and decapeptide analogue of the gamma chain of fibrinogen bind to different sites on the platelet fibrinogen receptor,” Blood. 66 Suppl 1: 305a), which is cleaved by human leukocyte elastase (McDonald, J. A. et al., 1980, “Degradation of fibronectin by human leukocyte elastase,” J. Biol. Chem. 255: 8848-8858).

The assay compound, threonine-proline-Rho110 (Beckman Coulter, Inc.) was identified as a substrate for cathepsin C (dipeptidyl-peptidase I, EC 3.4.14.1) and cathepsin G (EC 3.4.21.19) by a screen of many different dipeptide derivatives. Cathepsin C (DPPI) is a lysosomal cysteine peptidase that is found in relative abundance in cytotoxic cells (Thiele, D. L. et al., 1990, “Mechanism of L-leucyl-L-leucine methyl ester-mediated killing of cytotoxic lymphocytes: Dependence on a lysosomal thiol protease, dipeptidyl peptidase I, that is enriched in these cells,” Proc. Natl. Acad. Sci. USA. 87: 83-87). Cathepsin G is a serine endopeptidase that is a major component of the azurophil granules of polymorphonuclear leukocytes. Cathepsin G activity is high in promonocytic cells, but reduced in mature monocytes (Hohn, P. A. et al., 1989, “Genomic organization and chromosomal localization of the human cathepsin G gene,” J. Biol. Chem. 264: 13412-13419.

Other suitable leaving groups are described in Table 1 of U.S. Pat. No. 5,698,411 (Lucas, et al.) and U.S. Pat. No. 5,976,822 (Landrum et al.), and include: (Acetyl-α-D-glucopyranosyl) Rho 110; (Adenine)₂ Rho 110; (Adenosine Monophosphate)₂ Rho 110; (Adenosine) Rho 110; (B-D-Galactopyranoside)₂ Rho 110; (B-D-glucuronide)₂ Rho 110; (Butyrl-Thiocholine)₂, (Cytosine)₂ Rho 110; (Guanine)₂ Rho 110; (H Gly)₂ Rho 110; (H Gly-Arg)₂ Rho 110; (H Gly-Gly-Arg)₂ Rho 110; (H Gly-Leu)₂ Rho 110; (H Gly-Phe-Gly-Ala)₂ Rho 110; (H Gly-Pro-Leu-Gly-Pro)₂ Rho 110; (H-Gly)₂ -4′chloro-Rho 110; (H-Gly)₂ Rho 110; (H-Gly-Ala-Ala-Ala)₂ Rho 110; (H-Gly-Arg)₂ Rho 110; (H-Gly-Gly-Arg)₂ Rho 110; (H-Gly-Pro)₂ Rho 110; (H-Gly-Pro-Leu-Gly-Pro) Rho 110; (Hippuryl-His-Leu)₂ Rho 110; (H-L Ala-Ala-Ala-Ala)₂ Rho 110; (H-L Ala-Pro)₂ Rho 110; (H-L Leu-Leu-Arg)₂ Rho 110; H-L Lys-Ala)₂ Rho 110; (H-L Lys-Ala)₂ Rho 110.Sulfo.4TFA; (H-L Lys-Ala-Lys-Ala)₂ Rho 110; (H-L Pro-Arg)₂ Rho 110; (H-L-Ala)₂ -4′chloro-Rho 110; (H-L-Ala)₂ -Rho 110; (H-L-Ala-Ala)₂ Rho 110; (H-L-Ala-Ala-Ala)₂ Rho 110; (H-L-Ala-Ala-Pro-Ala)₂ Rho 110; (H-L-Ala-Ala-Tyr)₂ Rho 110; (H-L-Ala-Arg-Arg)₂ Rho 110; (H-L-Ala-Gly)₂ Rho 110; (H-L-Ala-Phe-Lys)₂ Rho 110; (H-L-Ala-Pro)₂ -Rho 110; (H-L-Ala-Pro-Ala)₂ Rho 110; (H-L-Arg)₂ Rho 110; (H-L-Arg-Arg)₂ Rho 110; (H-L-Arg-Gly-Glu-Ser)₂ Rho 110; (H-L-Asp)₂ -Rho 110; (H-L-Cys)₂ -Rho 110; (H-L-Gln-Ser)₂ Rho 110; (H-L-Glu)₂ -Rho 110; (H-L-Glu-Cys-Gly)₂ Rho 110; (H-L-Glu-Gly-Arg)₂ Rho 110; (H-L-Glu-Gly-Phe)₂ Rho 110; (H-L-Glu-Lys-Lys)₂ Rho 110; (H-L-Gly-Arg)₂ -Rho 110; (H-L-Leu)₂ -4′chloro-Rho 110; (H-L-Leu)₂ Rho 110; (H-L-Leu-Gly)₂ Rho 110; (H-L-Leu-Gly-Leu-Gly)₂ Rho 110; (H-L-Leu-Leu-Arg)₂ Rho 110; (H-L-Lys)₂ Rho 110; (H-L-Lys)₂ -Rho 110; (H-L-Lys-Ala)₂ -Rho 110; (H-L-Lys-Ala)₂ Rho 110-Sulfo; (H-L-Lys-Ala-Arg-Val)₂ Rho 110; (H-L-Lys-Ala-Arg-Val-Phe)₂ Rho 110; (H-L-Lys-Ala-Lys-Ala)₂ -Rho 110.6TFA; (H-L-Lys-Pro)₂ Rho 110; (H-L-Lys-Pro)₂ -Rho 110; (H-L-Met)₂ Rho 110; (H-L-Phe-Arg)₂ Rho 110; (H-L-Pro)₂ Rho 110; (H-L-Pro)₂ -Rho 110; (H-L-Pro-Arg)₂ Rho 110; (H-L-Pro-Phe-Arg)₂ Rho 110; (H-L-Ser)₂ Rho 110; (H-L-Serine Phosphate)₂ Rho 110; (H-L-Threonine Phosphate)₂ Rho 110; (H-L-Thr-Pro)₂ Rho 110; (H-L-thyroxine)₂ Rho 110; (H-L-Tyrosine Phosphate)₂ Rho 110; (H-L-Val-Leu-Lys)₂ Rho 110; (H-L-Val-Lys-Val-Lys)₂ Rho 110; (H-L-Val-Pro-Arg)₂ Rho 110; (H-L-Val-Ser)₂ Rho 110; (H-Pro-Arg)₂ -Rho 110; (N-Acetyl MET)₂ Rho 110; (N-Acetyl-L-Ala)₂ FL; (Phosphatidyl-choline)₂ Rho 110; (Saturated Hydrocarbon)₂ Rho 110; (Thymidine)₂ Rho 110; (Triacetin)₂ Rho 110; (Unsaturated Hydrocarton)₂ Rho 110; (Z-Ala-Ala)₂ Rho 110; (Z-Ala-Gly)₂ Rho 110; (Z-Thr-Pro)₂ Rho 110; (γ-Glu)₂ Rho 110; FL(Acetyl-Choline)₂; FL(butyrate)₂; FL(chloroacetate)₂; FL(chlorobutyrate)₂; FL(choline)₂; FL(heptanoate)₂; FL(hexanoate)₂; FL(palmitate)₂; FL(phosphate)₂; FL(propionate)₂; FL(valerate)₂; Fluorescein (acetate)₂; H-L-Leu Rhodol; H-L-Leu Rhodol; Rho 110 (phosphate)₂; Rho 110 (Phosphatidyl-choline)₂; Rho 110 (Phosphatidylinositol)₂; and Rho 110(AMP)₂.

Leaving groups for saccharidases are preferably prepared by the synthesis of monosaccharides, oligosaccharides or polysaccharides comprising between one and about ten sugar residues of the D-configuration. Examples of useful sugars are monosaccharides-pentoses; ribose; deoxyribose; hexose: glucose, dextrose, galactose; oligosaccharides-sucrose, lactose, maltose and polysaccharides like glycogen and starch. The sugar can be an alpha or beta configuration containing from 3 to 7 and preferably 5 to 6 carbon atoms. Analogs of these sugars can also be suitable for the invention. Preferably, the D-configuration of the monosaccharide or disaccharide is utilized. The monosaccharide or disaccharide can be natural or synthetic in origin.

Leaving groups for nucleases, nucleotidases, and nucleosidases are preferably prepared by the synthesis of nucleic acids, purines, pyrimidines, pentose sugars (i.e., ribose and deoxyribose) and phosphate ester. Examples are adenine, guanine, cytosine, uracil and thymine. Leaving groups for restriction enzymes would include polynucleotides. The nucleic acids contain a purine or pyrimidine attached to a pentose sugar at the 1-carbon to N-9 purine or N-1 pyrimidine. A phosphate ester is attached to the pentose sugar at the 5′ position. Analogs of these building blocks can also be used.

Leaving groups for lipases are preferably prepared by the synthesis of simple lipids, compound lipids or derived lipids. Simple lipids can be esters of fatty acids, triglycerides, cholesterol esters and vitamin A and D esters. Compound lipids can be phospholipids, glycolipids (cerebrosides), sulfolipids, lipoproteins and lipopolysaccharides. Derived lipids can be saturated and unsaturated fatty acids and mono or diglycerides. Analogs of these lipids can also be used. Examples of lipids are: triglycerides—triolein, fatty acids—linoleic, linolenic and arachidonic; sterols—testosterone, progesterone, cholesterol; phospholipids-phosphatidic acid, lecithin, cephalin (phosphatidyl ethanolamine) sphingomyleins; glycolipids—cerebosides, gangliosides.

Leaving groups for esterases are preferably prepared by the synthesis of carboxylic acids comprising between 2 and 30 carbon atoms. The carboxylic acids can be saturated or unsaturated. The carboxylic acid preferably contains 2 to 24 carbons and more preferably 4 to 24 carbon atoms. Analogs of theses carboxylic acids can also be used. The carboxylic acids can be natural or synthetic in origin. Examples are butyric, caproic, palmitic, stearic, oleic, linoleic and linolenic.

Leaving groups for phosphatases are preferably prepared by the synthesis of phosphates, phosphatidic acids, phospholipids and phosphoproteins. Analogs of these compounds can also be used. Examples are ATP, ADP, AMP and cyclic AMP (c-AMP).

Leaving groups for peptidases are preferably prepared by the synthesis of peptides comprising between one and about ten amino acid residues of the L-configuration. Typically, it has been found that the synthesis of peptides having more than about six amino acids produces a low yield. However, where the yield is acceptable, peptides of greater length can be employed. The amino acids preferably contain 2-10 and preferably 2-8 carbon atoms. Analogs of these amino acids can also be suitable for the invention. If the amino acids are chiral compounds, then they can be present in the D- or L-form or also as a racemate. Preferably, the L-configuration of the amino acid is utilized. The amino acids of the oligopeptide can be natural and/or of synthetic origin. Amino acids of natural origin, such as occur in proteins and peptide antibiotics, are preferred. Synthetic amino acids can also be used, such as pipecolic acid, cyclohexylalanine, phenylglycine, alpha.-aminocyclohexylcarboxylic acid, hexahydrotyrosine, norleucine, or ethionine.

Suitable methods for synthesizing, purifying, and preparing such compounds are described in U.S. Pat. No. 5,698,411 (Lucas, et al.) and U.S. Pat. No. 5,976,822 (Landrum et al.), herein incorporated by reference.

### E. Preferred Methods for Assay Signal Detection

In accordance with the methods of the present invention, the detectable signal may be detected with a charge-coupled device (CCD) camera or similar detector capable of detecting and storing images resulting from the detected signal. Suitable CCD cameras are available from Alpha-Innotech (San Leandro, Calif.), Stratagene (La Jolla, Calif.), and BioRad (Richmond, Calif.), and Beckman-Coulter, Inc. (Fullerton, Calif.). The RavidVue™ (Beckman-Coulter, Inc.) particle shape and size analyzer may be employed for this purpose.

For the automated handling and processing of multiple samples, the SAGIAN™ Automated Assay Optimization™ System (Beckman-Coulter, Inc.), or the FLUOstar 97™ or POLARstar™ System (BMG), adapted to detect and store images with a CCD camera may be used. The SAGIAN™ Automated Assay Optimization™ System employs a Biomek® 2000 Laboratory Automation Workstation (Beckman-Coulter, Inc.) with BioWorks™ 3.1 Software (Beckman-Coulter, Inc.). Automation of the assay can be accomplished using SAGIAN AAO™ Software (Beckman-Coulter, Inc.) and a computer with Windows® NT 4.0 SP3 and Excel 97 (Microsoft Corporation). Fluorescence can be quantified using ImaGene 4.0 assay quantitation software (BioDiscovery Inc.). The FLUOstar 97™/POLARstar™ System is a fully automated microplate-based fluorescence reader developed to measure data on a vast array of fluorescence assays. Measuring from above or below the microplate enables both tissue culture and FIA applications. The POLARstar can detect definitive receptor binding results through fluorescence polarization readings with 384-well microplates.

Other software (e.g., LEADseeker, etc.) may alternatively be used to facilitate very rapid analysis of high density formats and permit the ultra-high throughput screening of a range of biological assays (Fowler A., et al, “A multi-modality assay platform for ultra-high throughput screening,” Curr. Pharm. Biotechnol. 2000 Nov;1(3):265-81).

Most preferably, however, flow cytometry methods will be employed to detect the detectable label. Flow cytometry involves the use of one or more beams of laser light projected through a liquid stream that contains particles, which when struck by the focused light generate signals that can be detected by detectors. These signals are then converted for computer storage and data analysis. By using multiple laser beams to illuminate the particle, and/or multiple wavelength selective detectors to detect light emitted from the particle, it is possible to distinguish different labels. In bead-based multiplexing assays run on cytometers, the label is usually a fluorescent dye. The amount of dye on each bead is measured as the beads flow individually past an optical detection point.

Methods of, and instrumentation for, flow cytometry are known in the art. Flow cytometry, in general, concerns the passage of a suspension of microparticles as a stream past electro-optical sensors, in such a manner that only one particle at a time passes the sensors. As each particle passes the sensors, the particle produces a signal due to light scattering, fluorescence, etc., the nature and amplitude of the signal varying with label bound to the particle. Descriptions of instrumentation and methods for flow cytometry are found in the literature (see, McHugh, “Flow Microsphere Immunoassay for the Quantitative and Simultaneous Detection of Multiple Soluble Analytes, “Methods in Cell Biology 42, Part B (Academic Press, 1994); McHugh et al., “Microsphere-Based Fluorescence Immunoassays using Flow Cytometry Instrumentation, “Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); Horan et al., “Fluid Phase Particle Fluorescence Analysis: Rheumatoid Factor Specificity Evaluated by Laser Flow Cytophotometry, “Immunoassays in the Clinical Laboratory, 185-189 (Liss 1979); Wilson et al., “A New Microsphere-Based Immunofluorescence Assay Using Flow Cytometry, “J. Immunol. Meth. 107: 225-230 (1988); Fulwyler et al., “Flow Microsphere Immunoassay for the Quantitative and Simultaneous Detection of Multiple Soluble Analytes, “Meth. Cell Biol. 33: 613-629 (1990); UK Patent No. 1,561,042 (Coulter Electronics Inc.); and Steinkamp et al., Review of Scientific Instruments 44(9): 1301-1310 (1973)).

All publications and patents mentioned in this specification are herein incorporated by reference to the same extent as if each individual publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for assaying one or more target analytes in a sample, wherein said method comprises:
(A) providing, for at least one target analyte to be assayed, a binding ligand of said target analyte, said binding ligand being bound to a solid support; wherein the ability of said binding ligand to bind to said target analyte is hindered by a steric interference that does not hinder the binding of all other target analyte(s) to all other binding ligand(s);
(B) determining, for such target analyte(s), the presence, absence, activity or concentration of said target analyte(s), by determining the extent of binding between said target analyte and said solid-support-bound binding ligand of said target analyte.

2. The method of claim 1, wherein said steric interference is provided by said solid support.

3. A method for assaying one or more target analytes in a sample, wherein said method comprises:
(A) providing, for at least one target analyte to be assayed, a binding ligand of said target analyte, said binding ligand being bound to a solid support; wherein said support is porous and wherein binding ligand is bound to said support within the pores of said support and said pores sterically interfere with the ability of said binding ligand to bind to said target analyte and wherein the ability of said binding ligand to bind to said target analyte is hindered by a steric interference that does not hinder the binding of all other target analyte(s) to all other binding ligand(s);
(B) determining, for such target analyte(s), the presence, absence, activity or concentration of said target analyte(s), by determining the extent of binding between said target analyte and said solid-support-bound binding ligand of said target analyte.

4. The method of claim 3, wherein said support is controlled pore glass or a porous polymeric material.

5. A method for assaying one or more target analytes in a sample, wherein said method comprises:
(A) providing, for at least one target analyte to be assayed, a binding ligand of said target analyte, said binding ligand being bound to a solid support; wherein said support comprises bound interfering molecules that sterically interfere with the ability of said binding ligand to bind to said target analyte but does not hinder the binding of all other target analyte(s) to all other binding ligand(s);
(B) determining, for such target analyte(s), the presence, absence, activity or concentration of said target analyte(s), by determining the extent of binding between said target analyte and said solid-support-bound binding ligand of said target analyte.

6. A method for assaying one or more target analytes in a sample, wherein said method comprises:
(A) providing, for at least one target analyte to be assayed, a binding ligand of said target analyte, said binding ligand being bound to a solid support; wherein the ability of said binding ligand to bind to said target analyte is hindered by a chemical interference that does not hinder the binding of all other target analyte(s) to all other binding ligand(s);
(B) determining, for such target analyte(s), the presence, absence, activity or concentration of said target analyte(s), by determining the extent of binding between said target analyte and said solid-support-bound binding ligand of said target analyte.

7. The method of claim 6, wherein said chemical interference is provided by said solid support.

8. The method of claim 6, wherein said support comprises a plasticized organic phase particle, and wherein said binding ligand is immobilized within the confines of such particle.

9. A method for assaying one or more target analytes in a sample, wherein said method comprises:
(A) providing, for at least one target analyte to be assayed, a binding ligand of said target analyte, said binding ligand being bound to a solid support; wherein said support comprises bound interfering molecules that chemically interfere with the ability of said binding ligand to bind to said target analyte but which do not hinder the binding of all other target analyte(s) to all other binding ligand(s);
(B) determining, for such target analyte(s), the presence, absence, activity or concentration of said target analyte(s), by determining the extent of binding between said target analyte and said solid-support-bound binding ligand of said target analyte.

10. The method of any of claims 5 or 9, wherein said interfering molecules hinder binding by presenting a partial barrier to binding by said target analyte.

11. The method of claim 10, wherein said interfering or competing molecules comprise a tethered chain of at least 5 carbon atoms.

12. The method of any of claims 1 or 6, wherein said determination of the extent of binding between a target analyte and a binding ligand of said solid support comprises incubating said solid support in the presence of a detectably labeled binding ligand-binding molecule and determining the presence, absence, or concentration of detectably labeled binding ligand-binding bound to said solid-support-bound binding ligand of said target analyte.

13. The method of claim 12, wherein said detectable label of said detectably labeled binding ligand-binding molecule is a fluorescent label.

14. The method of any of claim 12, wherein said determination of the extent of binding between said target analyte and said binding ligand of said solid support said step (B) employs flow cytometry.

15. A composition for assaying a target analyte, which comprises a binding ligand of said target analyte bound to a solid support, wherein said support provides a steric interference that hinders the ability of said target analyte to bind to said bound binding ligand.

16. The composition of claim 15, wherein said support is porous and wherein binding ligand is bound to said support within the pores of said support and said pores sterically interfere with the ability of said binding ligand to bind to said target analyte.

17. The composition of claim 16, wherein said support is controlled pore glass or a porous polymeric material.

18. A composition for assaying a target analyte, which comprises a binding ligand of said target analyte bound to a solid support, wherein said support provides a chemical interference that hinders the ability of said target analyte to bind to said bound binding ligand.

19. The composition of claim 18, wherein said support comprises a plasticized organic phase particle, and wherein said binding ligand is immobilized within the confines of such particle.

20. The composition of any of claims 15 or 18, wherein said support comprises bound interfering molecules that interfere with the ability of said binding ligand to bind to said target analyte.

21. The composition of claim 20, wherein said interfering molecules hinder binding by presenting a partial barrier to binding by said target analyte.

22. The composition of claim 21, wherein said interfering molecules comprise a tethered chain of at least 5 carbon atoms.

23. A kit for assaying a target analyte, which comprises:
(A) a first container containing a binding ligand of said target analyte bound to a solid support, wherein said support provides a steric or chemical interference that hinders the ability of said target analyte to bind to said bound binding ligand; and
(B) a second container containing a detectably labeled binding ligand-binding molecule.

24. The kit of claim 23, wherein said detectable label is a fluorescent label.
